(19) United States
(12) Patent Application Publication
(10) Pub. No.: US 2011/0092753 A1
(43) Pub. Date: Apr. 21, 2011

(54) HYDROISOMERIZATION AND SELECTIVE HYDROGENATION OF FEEDSTOCK IN IONIC LIQUID-CATALYZED ALKYLATION

(76) Inventors: Bi-Zeng Zhan, Albany, CA (US); Howard S. Lacheen, Richmond, CA (US); Hye Kyung C. Timken, Albany, CA (US)

(21) Appl. No.: 12/581,269

(22) Filed: Oct. 19, 2009

(51) Int. Cl. C07C 2/58

(52) U.S. Cl. 585/251

(57) ABSTRACT

A process for producing alkylate comprising contacting a first hydrocarbon stream comprising at least one olefin having from 2 to 6 carbon atoms which contains 1,3-butadiene and 1-butene with a hydroisomerization catalyst in the presence of hydrogen under conditions favoring the simultaneous selective hydrogenation of 1,3-butadiene to butenes and the isomerization of 1-butene to 2-butene and contacting the resulting stream and a second hydrocarbon stream comprising at least one isoparaffin having from 3 to 6 carbon atoms with an acidic ionic liquid catalyst under alkylation conditions to produce an alkylate is disclosed.

### FIELD OF THE INVENTION

The present invention relates to a process for the alkylation of light isoparaffins with olefins using a catalyst comprising an ionic liquid.

### BACKGROUND OF THE INVENTION

In general, conversion of light paraffins and light olefins to more valuable cuts is very lucrative to the refining industries. This has been accomplished by alkylation of paraffins with olefins, and by polymerization of olefins. One of the most widely used processes in this field is the alkylation of isobutane with C₃ to C₅ olefins to make gasoline cuts with high octane number using sulfuric and hydrofluoric acids. This process has been used by refining industries since the 1940's. The process was driven by the increasing demand for high quality and clean burning high-octane gasoline.

Alkylate gasoline is an efficient burning gasoline that constitutes about 14% of the gasoline pool. Alkylate gasoline is typically produced by alkylating isobutane with low-end olefins (mainly butenes). Currently, alkylate is produced by using HF and H₂SO₄ as catalysts. Although these catalysts have been successfully used to economically produce the best quality alkylate, the need for safer and environmentally friendlier catalysts systems has become an issue to the industries involved.

The quest for an alternative catalytic system to replace the current environmentally unfriendly catalysts has been the subject of varying research groups in both academic and industrial institutions. Unfortunately, thus far, no viable replacement to the current processes has been put into practice at commercial refineries.

Ionic liquids are liquids that are composed entirely of ions. The so-called “low temperature” ionic liquids are generally organic salts with melting points under 100 degrees C., often even lower than room temperature. Ionic liquids may be suitable for example for use as a catalyst and as a solvent in alkylation and polymerization reactions as well as in dimerization, oligomerization acetylation, metatheses, and copolymerization reactions.

One class of ionic liquids is fused salt compositions, which are molten at low temperature and are useful as catalysts, solvents and electrolytes. Such compositions are mixtures of components which are liquid at temperatures below the individual melting points of the components.

Ionic liquids can be defined as liquids whose make-up is entirely comprised of ions as a combination of cations and anions. The most common ionic liquids are those prepared from organic-based cations and inorganic or organic anions. The most common organic cations are ammonium cations, but phosphonium and sulphonium cations are also frequently used. Ionic liquids of pyridinium and imidazolium are perhaps the most commonly used cations. Anions include, but not limited to, BF₄⁻, PF₆⁻, haloaluminates such as Al₂Cl₇⁻ and Al₂Br₇⁻, [(CF₃SO₂)₂N)]⁻, alkyl sulphates (RSO₃⁻), carboxylates (RCO₂⁻) and many other. The most catalytically interesting ionic liquids for acid catalysis are those derived from ammonium halides and Lewis acids (such as AlCl₃, TiCl₄, SnCl₄, FeCl₃ . . . etc). Chloroaluminate ionic liquids are perhaps the most commonly used ionic liquid catalyst systems for acid-catalyzed reactions.

Examples of such low temperature ionic liquids or molten fused salts are the chloroaluminate salts. Alkyl imidazolium or pyridinium chlorides, for example, can be mixed with aluminum trichloride (AlCl₃) to form the fused chloroaluminate salts. The use of the fused salts of 1-alkylpyridinium chloride and aluminum trichloride as electrolytes is discussed in U.S. Pat. No. 4,122,245. Other patents which discuss the use of fused salts from aluminum trichloride and alkylimidazolium halides as electrolytes are U.S. Pat. Nos. 4,463,071 and 4,463,072.

U.S. Pat. No. 5,104,840 describes ionic liquids which comprise at least one alkylaluminum dihalide and at least one quaternary ammonium halide and/or at least one quaternary ammonium phosphonium halide; and their uses as solvents in catalytic reactions.

U.S. Pat. No. 6,096,680 describes liquid clathrate compositions useful as reusable aluminum catalysts in Friedel-Crafts reactions. In one embodiment, the liquid clathrate composition is formed from constituents comprising (i) at least one aluminum trihalide, (ii) at least one salt selected from alkali metal halide, alkaline earth metal halide, alkali metal pseudohalide, quaternary ammonium salt, quaternary phosphonium salt, or ternary sulfonium salt, or a mixture of any two or more of the foregoing, and (iii) at least one aromatic hydrocarbon compound.

Light isoparaffins (iC₃-iC₆) can be alkylated with light olefins (C₂⁻-C₅⁻) using acidic ionic liquid catalysts (and in other alkylation processes) to make high octane and clean burning alkylate gasoline. The use of 2-butenes and isobutylene as alkylation olefin feed stocks tend to produce a much higher quality alkylates than 1-butene feed stock. This is due the nature of the alkylation chemistry with isobutylene and 2-butene which tends to produce the highly desired clean burning alkylates of trimethyl pentanes. Whereas, alkylations with 1-butene tend to produce the less desirable alkylates of dimethyl hexanes. Similarly, alkylation of isobutane with 1-pentene tends to produce the less desirable alkylates than with 2-pentene.

Moreover, refinery olefin feeds also contain 1,3-butadiene (diene) in amounts of up to 2 wt %. The diene is much more reactive than C₄⁻ olefins and promotes oligomerization to form conjunct polymers in the alkylation process, which deactivate the ionic liquid catalyst leading to production of poorer alkylate. So, the presence of diene in the feedstock to the ionic liquid catalyzed alkylation deteriorates the alkylation efficiency and generates considerable undesirable polymer side products.

### SUMMARY OF THE INVENTION

The present invention relates to a process for producing alkylate comprising contacting a first hydrocarbon stream comprising at least one olefin having from 2 to 6 carbon atoms which contains 1-butene and 1,3-butadiene with an isomerization catalyst in the presence of hydrogen under conditions favoring the isomerization of 1-butene to 2-butene and the with simultaneous selective hydrogenation of 1,3-butadiene to butenes and contacting the isomerized stream and a second hydrocarbon stream comprising at least one isoparaffin having from 3 to 6 carbon atoms with an acidic ionic liquid catalyst under alkylation conditions to produce an alkylate stream.

### DETAILED DESCRIPTION OF THE INVENTION

The present invention relates to an alkylation process comprising contacting a hydrocarbon mixture comprising at least one olefin having from 2 to 6 carbon atoms and at least one isoparaffin having from 3 to 6 carbon atoms with an acidic ionic liquid catalyst under alkylation conditions. In one embodiment, the at least one olefin stream contains 1, 3 butadiene and 1-butene. In one embodiment at least a portion of the 1,3-butadiene is hydrogenated to form butenes and at least a portion of the 1-butene is simultaneously isomerized in the presence of hydrogen to 2-butene before alkylation. For olefin feed containing C₅ olefins, this isomerization process would convert 1-pentene to 2-pentene.

One component of a feedstock is at least one isoparaffin having from 3 to 6 carbon atoms. This component may, for example, be any refinery hydrocarbon stream which contains isoparaffins.

Another component of a feedstock is at least one olefin having from 2 to 6 carbon atoms. This component may, for example, be any refinery hydrocarbon stream which contains olefins. In one embodiment, the feedstock is a C₄ olefin stream. Refinery streams containing butenes which may be used as the feed stocks for alkylation typically contain up to 25% 1-butene of the total volume of the olefins in the stream. Refinery stream also contain up to 2% 1,3-butadiene. The 1,3-butadiene is much more reactive than C₄⁼ olefins and promotes oligomerization to form conjunct polymers in the alkylation process. Conjunct polymers deactivate the ionic liquid catalyst leading to formation of alkylate with poor quality. So, the presence of 1,3-butadiene in the feedstock to the ionic liquid catalyzed alkylation deteriorates the alkylation efficiency and generates considerable amounts of undesirable polymer side products.

The processes according to the present invention are not limited to any specific feedstocks and are generally applicable to the alkylation of C₃-C₆ isoparaffins with C₂-C₆ olefins from any source and in any combination.

In one embodiment, at least a portion of the olefin feedstock, which contains 1-butene and 1-3-butadiene, is contacted with a catalyst in the presence of hydrogen to isomerize 1-butene to 2-butene and to hydrogenate butadiene. Hydrogenation of 1,3-butadiene to butenes and isomerization of 1-butene to 2-butene can be achieved simultaneously, in one embodiment, by passing the refinery olefin feed stock containing 1-butene and 1,3-butadiene over alumina supported Pd catalyst. As noted above, isomerization of 1-butene to 2-butene and 1-pentene to 2-pentene makes a better feed stock for an ionic liquid catalyzed alkylation with isobutane and other isoparaffins for making high quality, clean burning and high octane alkylate gasoline. And removal of butadiene retards deactivation of the ionic liquid catalyst.

In the alkylation of isobutane with 2-butene and isobutylene in ionic liquids, for example, the produced alkylates have an octane number that is usually in the high 90s. However, the alkylation of isobutane with 1-butene in ionic liquids leads to alkylates with lower octane numbers of around 70.

Processes and catalysts for the hydroisomerization of olefinic hydrocarbons and the selective hydrogenation of dienes are well known in the art.

U.S. Pat. No. 4,132,745 discloses a process for isomerization of 1-butene to give 2-butenes with simultaneous hydrogenation of small butadiene quantities present in the reactant. A sulfurized palladium catalyst is used.

Catalysts containing Pd highly dispersed on alumina may be used for hydroisomerization. In one embodiment, 0.5 wt % Pd dispersed on alumina is used as a hydroisomerization catalyst. Supported catalysts which may be used comprise at least one metal of the eighth transition group of the Periodic Table. A preferred metal is palladium. The metal concentrations are in the range from 0.05 to 2.0 wt % (based on the complete catalyst), preferably from 0.1 to 1.0%. Useful support materials include MgO, Al₂O₃, SiO₂, TiO₂, SiO₂/Al₂O₃, CaCO₃ or activated carbon.

As an alternative to a process using a fixed bed reactor, double bond hydroisomerization can be practiced in a catalytic distillation reactor. In U.S. Pat. No. 6,242,661, isobutene and isobutane are removed from a mixed C₄ hydrocarbon stream which also contains 1-butene, 2-butene and small amounts of butadiene. A catalytic distillation process is used in which a particulate supported palladium oxide catalyst isomerizes 1-butene to 2-butene. Isomerization is desired because 2-butene can be separated from isobutene more easily than 1-butene. As 2-butene is produced, it is removed from the bottom of the column, upsetting the equilibrium and allowing for a greater than equilibrium amount of 2-butene to be produced. Butadiene in the feed stream is hydrogenated to butene.

In a double bond hydroisomerization process, hydrogen must be co-fed with the C₄ stream in order to keep the catalyst active. However, as a result, some of the butenes are saturated. This undesirable reaction leads to loss of valuable 2-butene feed for alkylation. It would be useful to develop an isomerization process in which the saturation rate of butenes to butanes is minimized.

Hydrogen is essential for the hydroisomerization over the catalyst. It is needed to partially hydrogenate diene to mono-olefin in stoichiometric quantity. Hydrogen is also required for the isomerization reaction though it is not consumed. For refinery C₄ olefin, the quantity of hydrogen required is H₂/diene molar ratio of 0.1 to 100, or 0.5 to 20, or 1.0 to 10 to achieve less than 5% 1-butene in C₄ olefin in the hydroisomerized product. Extra quantity of hydrogen may result in an exotherm leading to temperature rising of catalyst bed and in a loss of olefins because of their hydrogenation to paraffins over the catalyst. Lack of hydrogen leads to poor isomerization of 1-butene to 2-butene.

Uniform H₂ distribution throughout the catalyst bed is desirable to maintain the catalyst activity. Poor H₂ distribution in the reactor bed may lead to premature aging of the catalyst or poorly hydroisomerized olefin stream resulting poorer alkylate quality.

Thermodynamic data indicate that low temperature favors conversion of 1-butene to 2-butene. To ensure a complete conversion of dienes to mono-olefin, the reaction is carried out at 80° F. (26° C.) to 600° F. (315° C.), or 100° F. (38° C.) to 500° F. (260° C.), or at 110° F. (43° F.) to 400° F. (204° C.).

The reaction is carried out under pressure to keep olefin feed in liquid phase and hydrogen with the catalyst and to promote selective diene hydrogenation. The pressure is important to ensure homogeneous distribution of H₂ on catalyst surface preventing premature aging of the catalyst. The pressure depends on the composition of refinery olefin. Its range is 50 psi (5.5 bar) to 1000 psi (68.0 bar), or 90 psi (12.4 bar) to 800 psi (55.2 bar).

The reaction is carried out at varying space velocity. The LHSV is 0.1 to 30 h⁻¹, in particular, 0.5 to 20 h⁻¹, or 1 to 10 h⁻¹.

The catalyst is regenerated in-situ by hydrogen at a temperature range of 200° F. (93° C.) to 1000° F. (538° C.), or 300° F. (149° C.) to 800° F. (427° C.), and at a pressure range of 50 psi (5.5 bar) to 1000 psi (68.0 bar), or 90 psi (12.4 bar) to 800 psi (55.2 bar) for 0.5 to 48 hours, or 2 to 24 hours.

After simultaneous hydroisomerization and selective hydrogenation of the olefin-containing stream, a mixture of hydrocarbons as described above is contacted with a catalyst under alkylation conditions. A catalyst in accordance with the present invention comprises at least one acidic halide-based ionic liquid and may optionally include an alkyl halide or hydrogen chloride promoter. The present process is being described and exemplified with reference certain specific ionic liquid catalysts, but such description is not intended to limit the scope of the invention. The processes described may be conducted using any acidic ionic liquid catalysts by those persons having ordinary skill based on the teachings, descriptions and examples included herein.

The specific examples used herein refer to alkylation processes using ionic liquid systems, which are amine-based cationic species mixed with aluminum chloride. In such systems, to obtain the appropriate acidity suitable for the alkylation chemistry, the ionic liquid catalyst is generally prepared to full acidity strength by mixing one molar part of the appropriate ammonium chloride with two molar parts of aluminum chloride. The catalyst exemplified for the alkylation process is a 1-alkyl-pyridinium chloroaluminate, such as 1-butyl-pyridinium heptachloroaluminate.

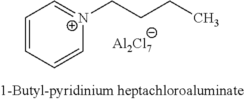

In general, a strongly acidic ionic liquid is necessary for paraffin alkylation, e.g. isoparaffin alkylation. In that case, aluminum chloride, which is a strong Lewis acid in a combination with a small concentration of a Broensted acid, is a preferred catalyst component in the ionic liquid catalyst scheme.

As noted above, the acidic ionic liquid may be any acidic ionic liquid. In one embodiment, the acidic ionic liquid is a chloroaluminate ionic liquid prepared by mixing aluminum trichloride (AlCl₃) and a hydrocarbyl substituted pyridinium halide, a hydrocarbyl substituted imidazolium halide, trialkylammonium hydrohalide or tetraalkylammonium halide of the general formulas A, B, C and D, respectively,

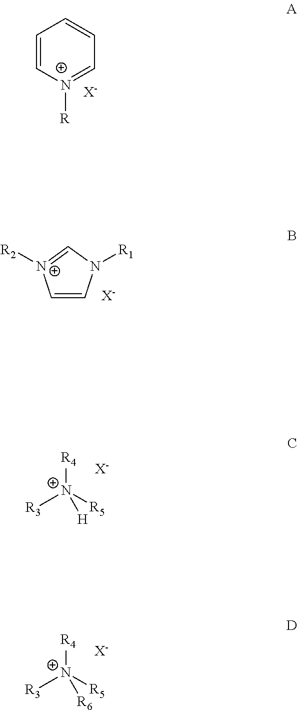

where R═H, methyl, ethyl, propyl, butyl, pentyl or hexyl group and X is a haloaluminate and preferably a chloroaluminate, and R₁ and R₂═H, methyl, ethyl, propyl, butyl, pentyl or hexyl group and where R₁ and R₂ may or may not be the same, and R₃, R₄, and R₅ and R₆=methyl, ethyl, propyl, butyl, pentyl or hexyl group and where R₃, R₄, R₅ and R₆ may or may not be the same.

The acidic ionic liquid is preferably selected from the group consisting of 1-butyl-4-methyl-pyridinium chloroaluminate, 1-butyl-pyridinium chloroaluminate, 1-butyl-3-methyl-imidazolium chloroaluminate and 1-H-pyridinium chloroaluminate.

In a process according to the invention an alkyl halide may optionally be used as a promoter.

The alkyl halide acts to promote the alkylation by reacting with aluminum chloride to form the prerequisite cation ions in similar fashion to the Friedel-Crafts reactions. The alkyl halides that may be used include alkyl bromides, alkyl chlorides and alkyl iodides. Preferred are isopentyl halides, isobutyl halides, butyl halides, propyl halides and ethyl halides. Alkyl chloride versions of these alkyl halides are preferable when chloroaluminate ionic liquids are used as the catalyst systems. Other alkyl chlorides or halides having from 1 to 8 carbon atoms may be also used. The alkyl halides may be used alone or in combination.

A metal halide may be employed to modify the catalyst activity and selectivity. The metal halides most commonly used as inhibitors/modifiers in aluminum chloride-catalyzed olefin-isoparaffin alkylations include NaCl, LiCl, KCl, BeCl₂, CaCl₂, BaCl₂, SrCl₂, MgCl₂, PbCl₂, CuCl, ZrCl₄ and AgCl, as described by Roebuck and Evering (Ind. Eng. Chem. Prod. Res. Develop., Vol. 9, 77, 1970). Preferred metal halides are CuCl, AgCl, PbCl₂, LiCl, and ZrCl₄.

HCl or any Broensted acid may be employed as co-catalyst to enhance the activity of the catalyst by boasting the overall acidity of the ionic liquid-based catalyst. The use of such co-catalysts and ionic liquid catalysts that are useful in practicing the present invention is disclosed in U.S. Published Patent Application Nos. 2003/0060359 and 2004/0077914. Other co-catalysts that may be used to enhance the activity include IVB metal compounds preferably IVB metal halides such as ZrCl₄, ZrBr₄, TiCl₄, TiCl₃, TiBr₄, TiBr₃, HfCl₄, HfBr₄ as described by Hirschauer et al. in U.S. Pat. No. 6,028,024.

Due to the low solubility of hydrocarbons in ionic liquids, olefins-isoparaffins alkylation, like most reactions in ionic liquids is generally biphasic and takes place at the interface in the liquid state. The catalytic alkylation reaction is generally carried out in a liquid hydrocarbon phase, in a batch system, a semi-batch system or a continuous system using one reaction stage as is usual for aliphatic alkylation. The isoparaffin and olefin can be introduced separately or as a mixture. The molar ratio between the isoparaffin and the olefin is in the range 1 to 100, for example, advantageously in the range 2 to 50, preferably in the range 2 to 20. In a semi-batch system the isoparaffin is introduced first then the olefin, or a mixture of isoparaffin and olefin. Catalyst volume in the reactor is in the range of 1 vol % to 70 vol %, or 4 vol % to 50 vol %. Vigorous stirring is desirable to ensure good contact between the reactants and the catalyst. The reaction temperature can be in the range −40° C. to +150° C., preferably in the range −20° C. to +100° C. The pressure can be in the range from atmospheric pressure to 8000 kPa, preferably sufficient to keep the reactants in the liquid phase. Residence time of reactants in the vessel is in the range a few seconds to hours, preferably 0.5 min to 60 min. The heat generated by the reaction can be eliminated using any of the means known to the skilled person. At the reactor outlet, the hydrocarbon phase is separated from the ionic phase by decanting, then the hydrocarbons are separated by distillation and the starting isoparaffin which has not been converted is recycled to the reactor.

Typical alkylation conditions may include a catalyst volume in the reactor of from 2 vol % to 50 vol %, a temperature of from −10° C. to +100° C., a pressure of from 300 kPa to 2500 kPa, an isopentane to olefin molar ratio of from 2 to 16 and a residence time of 1 min to 1 hour.

In one embodiment of a process according to the present invention, high quality gasoline blending components of low volatility are recovered from the alkylation zone. Those blending components are then preferably blended into gasoline.

The following Examples are illustrative of the present invention, but are not intended to limit the invention in any way beyond what is contained in the claims which follow.

### Example 1

### Hydroisomerization of C₄ Olefin Feed

Table 1 shows the composition of refinery C₄ olefin feeds before and after hydroisomerization with a 0.5 wt % Pd/Al₂O₃ catalyst. It demonstrates the conversion of 1-butene to 2-butene with complete saturation of 1,3-butene by the hydroisomerization process. The 1-butene concentration in the hydroisomerized product is less than 5% in C₄ olefin with minor or no olefin loss

TABLE 1
Composition of C₄ olefin stream before and after hydroisomerization
Hydroisomerization conditions

| Catalyst Temperature, ° F. | — | 150 | — | 140 |
|---|---|---|---|---|
| Pressure, psig | — | 300 | — | 350 |
| WHSV, h⁻¹ | — | 3.2 | — | 0.9 |
| H₂/diene molar ratio | — | 3.9 | — | 24 |
| Composition of olefin, wt % | Feed | Product | Feed | Product |
| Propane | 2.0 | 2.0 | 2.1 | 2.1 |
| Propene | 1.2 | 1.0 | 0.7 | 0.3 |
| 1-Butene | 12.3 | 1.9 | 10.7 | 1.7 |
| trans-2-Butene | 14.8 | 23.7 | 18.1 | 25.3 |
| cis-2-Butene | 9.6 | 10.8 | 11.1 | 10.3 |
| iso-Butene | 13.4 | 13.3 | 20.5 | 19.0 |
| 1,3-Butadiene | 0.2 | 0 | 0.2 | 0 |
| n-Butane | 11.7 | 12.0 | 12.9 | 17.1 |
| iso-Butane | 31.7 | 31.6 | 22.0 | 21.0 |
| 1-butene in C4 olefin, % | 24.5 | 3.8 | 17.7 | 3.0 |

### Example 2

### Hydroisomerization of C₃ and C₄ Olefin Feed

Table 2 shows the composition of a refinery C₄ olefin feed stream containing rich propene before and after hydroisomerization with a 0.5 wt % Pd/Al₂O₃ catalyst. It demonstrates the conversion of 1-butene to 2-butene with complete saturation of 1,3-butene by hydroisomerization. The 1-butene in C₄ olefin was isomerized from 19.9% in the feed to about 6% in the product.

TABLE 2
Composition of C₄ olefin stream before and after hydroisomerization
Hydroisomerization conditions

| Catalyst Temperature, ° F. | — | 140 | 150 |
|---|---|---|---|
| Pressure, psig | — | 350 | 350 |
| WHSV, h⁻¹ | — | 2.5 | 2.5 |
| H₂/diene molar ratio | — | 4.7 | 4.7 |
| Composition of olefin stream, wt % | Feed | Product | Product |
| Propane | 0.8 | 1.3 | 1.2 |
| Propene | 16.6 | 16.5 | 15.9 |
| 1-Butene | 10.7 | 2.8 | 2.5 |
| trans-2-Butene | 12.3 | 18.3 | 19.5 |
| cis-2-Butene | 7.8 | 10.1 | 10.3 |
| iso-Butene | 11.3 | 11.0 | 11.3 |
| 1,3-Butadiene | 0.2 | 0 | 0 |
| n-Butane | 10.8 | 11.0 | 11.6 |
| iso-Butane | 26.9 | 25.7 | 26.5 |
| 1-butene in C4 olefin, % | 19.9 | 6.6 | 5.7 |

### Example 3

### Hydroisomerization of Refinery C₅ Olefin Feed

Table 3 shows the C₅ olefin composition before and after hydroisomerization over a 0.5 wt % Pd/Al₂O₃ catalyst. 1-Pentene is isomerized to 2-Pentene with over 80% conversion on the catalyst. 3-Methyl-1-butene and 2-methyl-1-butene are converted to 2-methyl-2-butene with about 50% conversion.

TABLE 3
Composition of C₅ olefin stream before and after hydroisomerization

| Hydroisomerization conditions | | | |
|---|---|---|---|
| Reactor temperature, ° F. | | 150 | 150 |
| Unit Pressure, psig | | 350 | 90 |
| WHSV, h⁻¹ | | 2.4 | 2.4 |
| H₂ flow (SCF/B-olefin feed) | | 16 | 16 |
| Composition of olefin, wt % | Feed | Product | Product |
| trans-2-Butene | 5 | 6.6 | 7.5 |
| 1-Butene | 0.7 | 0.5 | 0.8 |
| iso-Butene | 0.5 | 0.4 | 0.6 |
| cis-2-Butene | 6.5 | 3.5 | 4.5 |
| i-C5 | 46.3 | 47.5 | 45.0 |
| n-C5 | 4.1 | 4.9 | 4.3 |
| 3-methyl-1-Butene | 3.0 | 0.9 | 0.8 |
| trans-2-Pentene | 7.2 | 11.7 | 10.7 |
| 2-methyl-2-Butene | 7.4 | 12.7 | 11.9 |
| 1-Pentene | 4.1 | 0.7 | 0.6 |
| 2-methyl-1-Butene | 8.2 | 4.6 | 4.8 |
| cis-2-Pentene | 3.7 | 3.5 | 3.2 |

### Example 4

### Effect of 1-butene Concentration on the Alkylate Quality

Table 4 shows the alkylate quality in the alkylation of refinery C₄ olefin with C₄ isoparaffin over 1-butyl-pyridinium heptachloroaluminate catalyst. The alkylate quality significantly improved with olefin feed after hydroisomerization pretreatment. The RON increased from 89.0 with untreated C₄ olefin feed (31% 1-butene) to ˜95 with pretreated C₄ olefin feed (3% 1-butene). The heavy alkylation product (C₁₀+) is reduced from 10.6% to 5.8% by hydroisomerization for the removal of 1,3-butandiene.

TABLE 4
Alkylate product made with C₄ olefin feed with varying degree of
hydroisomerization (e.g. varying 1-butene concentration)

| | Before | After |
|---|---|---|
| Olefin Feed | hydroisomerization | hydroisomerization |
| 1-butene in C₄ olefin, % | 31 | 3 |
| 1,3-butadiene, % | 0.3 | 0 |
| Heavy product (C₁₀+), % | 10.6 | 5.8 |
| Alkylate quality | | |
| RON | 89.0 | 95.4 |
| MON | 89.8 | 92.5 |

### Example 5

### Effect of Conjunct Polymer in Ionic Liquid Catalyst on Alkylate Quality

Table 5 shows the effect of conjunct polymer accumulated in ionic liquid on the alkylate quality. The RON of alkylate decreases from 94-96 to 80-91 when conjunct polymer concentration in ionic liquid is over 20 wt %.

TABLE 5
The effect of conjunct polymer on alkylate quality

| | Conjunct polymer in ionic liquid catalyst | | |
|---|---|---|---|
| | (wt %) | | |
| Alkylate quality | 2-5 | 11 | 20 |
| RON | 94-96 | 94-95 | 89-91 |

There are numerous variations on the present invention which are possible in light of the teachings and supporting examples described herein. It is therefore understood that within the scope of the following claims, the invention may be practiced otherwise than as specifically described or exemplified herein.

What is claimed is:

1. A process for producing alkylate comprising contacting a first hydrocarbon stream comprising at least one olefin having from 2 to 6 carbon atoms which contains 1,3-butadiene and 1-butene with a hydroisomerization catalyst in the presence of hydrogen under conditions favoring the simultaneous selective hydrogenation of 1,3-butadiene to butenes and the isomerization of 1-butene to 2-butene and contacting the resulting stream and a second hydrocarbon stream comprising at least one isoparaffin having from 3 to 6 carbon atoms with an acidic ionic liquid catalyst under alkylation conditions to produce an alkylate.

2. A process according to claim 1, where the acidic ionic liquid is a chloroaluminate ionic liquid.

3. A process according to claim 2, wherein the acidic ionic liquid is selected from the group consisting of 1-butyl-4-methyl-pyridinium chloroaluminate (BMP), 1-butyl-pyridinium chloroaluminate (BP), 1-butyl-3-methyl-imidazolium chloroaluminate BMIM) and 1-H-pyridinium chloroaluminate (HP).

4. A process according to claim 1, wherein the isoparaffin is selected from the group consisting of isobutane, isopentanes and mixtures thereof.

5. A process according to claim 1, wherein the first hydrocarbon stream contains up to 2% 1,3-butadiene.

6. A process according to claim 1, wherein the alkylation conditions include a catalyst volume in the reactor of from 4 vol % to 50 vol %, a temperature of from −10° C. to 100° C., a pressure of from 300 kPA to 2500 kPa, an isopentane to olefin molar ratio of from 2 to 16 and a residence time of 1 minute to 1 hour.

7. A process according to claim 1, wherein the first hydrocarbon stream is a refinery C₄ olefin-containing stream.

8. A process according to claim 1, wherein the acidic ionic liquid catalyst further comprises an alkyl halide.

9. A process according to claim 8, where the alkyl halide is selected from the group consisting of methyl halide, ethyl halide, propyl halide, 1-butyl halide, 2-butyl halide, tertiary butyl halide, pentyl halides, isopentyl halide, hexyl halides, isohexyl halides, heptyl halides, isoheptyl halides, octyl halides and isooctyl halides.

10. A process according to claim 1, wherein the acidic ionic liquid is selected from the group consisting of 1-butyl-4-methyl-pyridinium chloroaluminate (BMP), 1-butyl-pyridinium chloroaluminate (BP), 1-butyl-3-methyl-imidazolium chloroaluminate BMIM) and 1-H-pyridinium chloroaluminate (HP).

11. A process according to claim 1, wherein the olefin stream contains up to 100% 1-butene.

12. The process for olefin alkylation of claim 1, wherein the hydroisomerization catalyst is a transition group metal dispersed over a support.

13. The process for olefin alkylation of claim 11, wherein the transition metal is selected from the group consisting of palladium, platinum, ruthenium, and nickel.
